(19) United States
(12) Patent Application Publication
(10) Pub. No.: US 2022/0211593 A1
(43) Pub. Date: Jul. 7, 2022

(54) Reflective Composition

(71) Applicant: THEUNSEEN LIMITED, London (GB)

(72) Inventors: Lauren Bowker, London (GB); Louise Anderson, London (GB)

(21) Appl. No.: 17/605,936

(22) PCT Filed: Apr. 24, 2020
(86) PCT No.: PCT/EP2020/061514

§ 371 Date: Oct. 22, 2021

(30) Foreign Application Priority Data
Apr. 24, 2019 (GB) 1905748.8

(51) Int. Cl. A61K 8/29; A61K 8/81; A61Q 1/02; A61Q 5/06

(52) U.S. Cl. CPC A61K 8/29; A61K 8/8141; A61Q 1/02; A61K 2800/436; A61K 8/8182; A61K 8/8135; A61K 2800/43; A61Q 5/065

(57) ABSTRACT

The invention provides a cosmetic composition for providing a retro-reflective effect to incident light to a natural or artificial substrate such as hair, skin, nails, eyelashes and the like. The composition comprises retroreflective microparticles and a transparent coating composition and is substantially free of goniochromatic colouring agents.

This invention relates to a reflective cosmetic composition for providing a retro-reflective effect to incident light, particularly to a cosmetic composition providing a retro-reflective effect and to the use of retro-reflective microparticles in a cosmetic composition for application to an artificial or keratinous substrate to provide a retro-reflective effect. The cosmetic composition is suitable for use on the face and body

Treating keratinous substrates such as hair, skin, eyelashes, lips and nails, to provide a visual effect, for example colouration, has been known since the earliest times using natural pigments and dyes and the global cosmetics industry provides myriad colour and effect products worldwide. A wide range of other visual effects such as providing highlights, reflection and shimmering effects to provide accentuation, embellishment, and the like are also known.

Many products are known for providing reflective effects on a keratinous substrate, including glitter sprays, iridescent materials for example mica and goniochromatic materials and the like. Goniochromism provides a reflective colour-change or shimmering effect such that a change in colour is observed as the angle of view or the angle of illumination of a surface changes and provides a multi-coloured effect.

U.S. Pat. No. 5,624,731 describes a multi-colour, multi-image reflective goniochromatic display provided by a multi-layer material which includes a base layer, an intermediate layer containing a pigment providing goniochromatic properties and a retro-reflectivity layer overlying the transparent intermediate layer and containing transparent beads where light reflected from the base layer or intermediate layer passes through the retro-reflectivity layer to enhance the brilliance of the reflected colours.

U.S. Pat. No. 7,767,214 describes a cosmetic composition containing a goniochromatic colouring agent and at least one further reflective microparticles which provides a certain level of reflectivity and provide a volumizing effect.

Retro-reflection is an effect which typically occurs when a surface reflects a large portion of directed incident light back to its source. Retro-reflective materials appear brightest to observers nearest the light source (such as a motorist). The object's brightness depends on the intensity of the light striking the object and the materials the object is made of.

Retro-reflective surfaces do not typically show different colours depending on the angle of view or illumination and typically provide an intense, bright, white reflected light, for example in cats eyes and retro-reflective paint used to provide illumination of objects in road safety applications.

We have now found that a high intensity of reflection of incident light in the direction towards the source of light may be obtained by providing a cosmetic composition which contains retro-reflective microparticles within a transparent or translucent coating composition which is substantially free of goniochromatic materials and which are acceptable for cosmetic use on hair and skin. We have also found that a retro-reflective effect may be secured in a “temporary” composition, removable from the surface or hair to which it is applied, by washing.

In a first aspect, the invention provides a cosmetic composition for providing a retro-reflective effect to incident light, comprising retro-reflective microparticles, preferably microspheres and a transparent or translucent coating composition and being substantially free of goniochromatic colouring agents.

The invention provides in a second aspect, use of retro-reflective microparticles in a cosmetic composition comprising a transparent or translucent coating composition and being substantially free of goniochromatic colouring agents, to provide a retro-reflective effect to incident light.

The composition is suitable for use in cosmetic applications and compliant with applicable European and US regulations for hair and skin products. The composition is preferably substantially free of heavy plasticisers, for example camphor and triphenylphosphate, and organic solvents employed in nail varnishes for example ethyl acetate and butyl acetate. The cosmetic composition is suitably a temporary cosmetic composition and may be removed from a substrate to which it is applied, for example hair, the face or the body, by standard washing, preferably, in one or more than one wash, preferably not more than 10 washes, more preferably not more than 5 washes, for example 2 or 3 washes.

In order to be removable from the substrate and so provide a temporary effect, the cosmetic composition is suitably an aqueous solution, aqueous emulsion (oil in water) or otherwise removable from the substrate to which it is applied using water and shampoo or other water soluble cleaning products for personal use.

The term “standard wash” means a normal, everyday washing exercise carried out for a typical period and under typical conditions by an average person, using typical washing products such as soap, shampoo or the like. The term “standard wash” also includes a typical make-up removal process with an average person using conventional products such as make-up remover, in a typical manner. In a further aspect, the invention provides a process of providing a substrate, preferably a keratinous substrate, with the capability of exhibiting a retro-reflective effect comprising applying a cosmetic composition according to the invention to the substrate. Suitably, the cosmetic composition provides a temporary capability of exhibiting a retro-reflective effect by being removable from the substrate by subjecting to standard washing, preferably being removable from the substrate in 5 or fewer standard washes.

The term “retro-reflectivity” as employed herein refers reflection of a large proportion of directed incident light back to its source with minimal scattering and typically is observed with materials which exhibit a high refractive index, preferably at least 1.3 and more preferably at least 1.7. Retro-reflective materials typically provide a white, silver or rainbow light and appear brightest to observers near the light source (such as a motorist). The object's brightness depends on being in-line with the direction of the incident light so as to reflect towards the light source. By contrast, a goniochromatic effect is generally due to interference effects in which incident light is partly reflected from the surface and partly refracted through the material making upon the surface. A paper “Appearance Measurement of Goniochromatic Colours” published in 2012 (Predicting Perceptions: Proceedings of the 3^{rd} International Conference on Appearance, pp 149-154, Edinburgh, UK, ISBN 978-1-4716-6869-2) sets out how the goniochromatic effect may be quantified.

Advantageously, cosmetic compositions according to the invention provide a retro-reflection effect with a high intensity reflection of incident light when applied to keratinous substrates, for example hair, skin, eyelashes and nails, or artificial substrates. The cosmetic composition is suitable for use on the face or body. The composition may be applied manually or by using an implement. The retro-reflectivity effect is particularly marked upon application of directional light to the composition, for example by flash photography.

Suitably the cosmetic composition may be applied to a substrate so as to form a thin-layer, The retro-reflective microparticles are suitably dispersed in the layer and are disposed at or near the surface of the layer. Suitably, the layer of the cosmetic composition has a thickness which does not impart the feel of the substrate to the touch. Preferably the layer of the cosmetic composition has a thickness of the same order of magnitude, and more preferably not more than 75% thicker or more than 50% thicker than the size of the microparticles such that a monolayer of microparticles is formed on deposition of the cosmetic composition on the substrate. Preferably the thin-layer has a thickness of from 0.1 to 100 microns, preferably not more than 60 microns.

Suitably, the amount of material of different refractive index to the microparticles that light reflected through the microparticles has to pass through is minimised so as to reduce the effects of scattering and refraction of light which reduce the intensity of the reflected light and its visual impact on the observer. A thin layer enables a higher proportion of incident light to be reflected back to the source than if a thicker layer with additional components in the composition were present. Such thicker layers absorb or diffract a proportion of the incident light which may reduce the intensity of the reflected light as compared to the intensity of reflected light when a thin layer of the cosmetic composition is employed on the surface of the substrate.

Preferably, the cosmetic composition has a uniform thickness upon application to the substrate so as to enable maximum reflectance of incident light on the substrate enhancing colour intensity and vibrancy and feel.

The cosmetic composition suitably possesses desirable “feel” or tactile properties upon application to the substrate, for example hair, such that it is pleasant or not irritating for the wearer and tactile and suitably feels natural to touch. Desirably, the composition or the substrate to which the composition is applied does not form clumps or lead to other characteristics which are disadvantageous or undesirable as regards visual or tactile impact.

The term “feel” as employed herein refers to the sensory perception of the substrate especially a keratinous substrate, with the cosmetic composition applied to it and includes appearance/visual and tactile characteristics, for example tackiness, shine, weight, width, movement of the substrate and the like in the context of its use.

The retro-reflective microparticles suitably comprise transparent or translucent microspheres. The microparticles are preferably a glass, or a metal for example aluminium, and more preferably comprise a metal titanate, especially barium titanate. In a preferred embodiment, the retro-reflective microparticles comprise glass beads doped with a metal titanate, for example barium titanate.

Suitably, the microparticles provide specular reflection in addition to retro-reflectivity. The term “specular reflection” as employed herein refers to reflection from a surface where the incident light is reflected into a single outgoing direction and provides a mirror-like reflectance effect. Preferably, the microparticles comprise a reflective coating or shell which provides specular reflection. The reflective coating suitably comprises a metal, preferably aluminium, or a metallic compound for example a metallic oxide. Suitably, the coating is hemispherical.

Suitably the retro-reflective microparticles are chemically inert facilitating their use in a cosmetic composition. Preferably the retro-reflective microparticles have a refractive index of at least 1.5, more preferably at least 1.6. Suitably, the retro-reflective microparticles have a refractive index which is less than 3 and desirably less than 2.5 as a higher refractive index may compromise the visual effect by appearing yellow to the observer. Optimally, the refractive index is from 1.7 to less than 2.0, for example 1.9.

Examples of suitable retro-reflective microparticles are set out in Table 1 below.

TABLE 1

| | | | | Hemispherical |
|---|---|---|---|---|
| | | | | aluminium |
| Trade name | Supplier | Particle size | | coating? |
| Starglow | Glowtec | 30-40 μm | | No |
| reflective Clear | | | | |
| Starglow | Glowtec | 30-40 μm | | Yes |
| reflective Ultra | | | | |
| Reflective | SFXC | 55-75 μm | | |
| aluminium beads | | | | |
| P2453BTA | Prizmalite | Average = | 45-50 μm | Yes |
| (no surface | | Maximum = | 100 μm | |
| treatment) | | | | |
| P2453BTA F-22 | Prizmalite | Average = | 45-50 μm | Yes |
| coated | | Maximum = | 100 μm | |
| (fluorochemically | | | | |
| coated) | | | | |
| P2453BTA | Prizmalite | Average = | 45-50 μm | Yes |
| RS2-100-9A | | Maximum = | 100 μm | |
| P2453BTA Silane | Prizmalite | Average = | 45-50 μm | Yes |
| coated | | Maximum = | 100 μm | |

*STARGLOW, GLOWTEC, SFXC, PRIZMALIT are trademarks.

Suitably, the retro-reflective microparticles are present in the cosmetic composition at a level of 1 to 80%, preferably 5 to 70% by weight of the composition. In one embodiment a composition comprising 30 to 40% retroreflective microparticles provides an optimal balance between retro-reflective properties and feel when applied to hair.

Suitably the retro-reflective microspheres have a particle size of less than 100 microns.

Known iridescent components employed in cosmetic compositions to provide a reflective effect for example “shimmering” or “glittering”, may typically have a particle size up to 200 microns. However, when employed in hair compositions, such particles having a particle size significantly greater than the typical width of hair strands may compromise the feel of the hair.

The particle size of the microspheres is selected according to the desired effect and the intended use, for example as a cosmetic hair composition, face composition or cosmetic body composition. Preferably, the particle size of the microspheres is from 0.1 to 100 microns, for example 1 to 70 microns and especially 20 to 50 microns. Advantageously, with a particle size of a similar order of magnitude to hair fibres, the hair retains a natural feel while providing a vibrant colour effect without compromising the “feel” of the hair.

The transparent or translucent coating composition suitably enables maximum reflectance of incident light, improving colour vibrancy and intensity and may be aqueous based or non-aqueous based, for example oil based and the base will be selected according to the desired final product form. The coating composition suitably comprises a thickening agent and/or a film-forming component. A thickening effect and film-forming effect may be provided by a single component which provides a thickening effect and a film-forming effect. The term “thickening agent” also includes a rheology or viscosity modifier.

The thickening agent in the coating composition is suitably selected from a cross-linked polyelectrolyte and a hydrophobically modified alkali swellable emulsion thickening product. Cross-linked polyelectrolytes are preferred. Suitably, the coating composition is film-forming such that the cosmetic composition is coatable onto a substrate, for example hair, to form a continuous film and adhere to the substrate.

Suitable cross-linked polyelectrolytes comprise a dissociating group in at least some, and desirably all of the monomeric units of the polymer, capable of dissociating in a polar and especially aqueous environment. Preferably the monomeric units comprise a group selected from —COOH, —SO₃H, —PO₃H₂ and —NH₂. Suitably, the cross-linked polyelectrolyte is able to form a swellable structure which swells on contact with the polar environment and desirably forms regions of micro-gels. Suitably the microgels have a size of 0.1 to 50, preferably 0.5 to 20, more preferably 1 to 10 microns, for example 3 to 4 microns so as to form around the retro-reflective microparticles and aid its suspension within the composition which is believed to improve the uniformity of deposition of the composition on the substrate.

The cross-linked polyelectrolyte may comprise a natural thickener and/or preferably a synthetic thickener. Examples of natural thickening agents include carboxymethyl cellulose, alginate, chitosan, pectin. Examples of suitable synthetic polyelectrolytes include polymers and copolymers comprising polyacrylic acid, polymethacrylic acid, polystyrene sulfonate and derivatives of these polymers and their salts. Examples of natural and synthetic polyelectrolytes are disclosed in “A comprehensive review on polyelectrolyte complexes”, Meka et al, Drug Discovery Today, Vol 22, No 11, November 2017.

Reference herein to acrylic or acrylate includes methacrylic and methacrylate mutatis mutandis unless otherwise stated.

Suitably, the cross-linked polyelectrolyte comprises a polymer which has been modified so as to comprise a hydrophilic region and a hydrophobic region. The thickening agent preferably is soluble in water at 25° C. at a level of at least 1% by weight, preferably at least 1.2% by weight and especially at least 1.5% by weight provided that the cosmetic composition enables a smooth application to the substrate without compromising the feel of the substrate. More preferably, the thickening agent is soluble in water at 25° C. at a level of 2% by weight.

Preferably, the thickening agent is selected from a polyacrylate polymer, cross-polymers and copolymers of (poly)acrylates and polyacrylic acids, for example polyacrylate crosspolymer-6-2-methyl propan-2-ol and acrylates/C10-30 alkyl acrylate crosspolymer, alkyl acrylates, urethane acrylates, hydrophilically modified starch, for example hydroxypropyl starch modified with a COOH, —SO₃H, —PO₃H₂ and —NH₂ group, polyvinyl pyrollidone/vinyl acetate copolymers, polyquaternium compounds and cetearyl alcohol, ceteareth-20.

Examples of suitable cross-linked polyelectrolytes include SEPIMAX ZEN available from Seppic, STRUCTURE XL and DYNAMX H₂O available from AkzoNobel, CARBOPOL and FIXATE FREESTYLE range of polymers available from Lubrizol including CARBOPOL® Ultrez 30, CARBOPOL® Ultrez 21, COVACRYL range of polymers available from Sensient including COVACRYL P12, COVACRYL E14 WP, COVACRYL A15 WP, LUVISKOL range of polymers available from BASF including LUVISKOL VA 64, LUVISKOL VA 37-E, TRlquat range of polymers available from Tri-K including TRIQUAT FORCE, CELQUAT range of polymers available from AkzoNobel including CELQUAT L-200. SEPIMAX, STRUCTURE, DYNAMX, CARBOPOL, FIXATE FREESTYLE, COVACRYL, LUVISKOL, TRIQUAT, CELQUAT are trademarks.

Where a hydrophobically modified alkali soluble polymer is employed as a thickening agent, the hydrophobically modified alkali soluble polymer is preferably an emulsion (HASE).

The hydrophobically modified alkali soluble polymer suitably comprises a polymer or a copolymer having one or more monomeric units derived from one or more of acrylic acid, methacrylic acid, hydroxyethyl methacrylic acid, alkyl preferably C₁ to C₄, methacrylic acid, alkyl preferably C₁ to C₄, acrylic acid, alkyl preferably C₁ to C₄, amino acrylic acids, acrylamides, esters of any such acrylic or methacrylic monomers and ethers of any such acrylic or methacrylic monomers, acrylated dimethiconol. Suitably the polymer is hydrophobically modified by a modified polyalkylene glycol having a mid to longer chain alcohol for example modified polyethylene glycol, modified polypropylene glycol and modified polybutylene glycol.

Preferably, the hydrophobically modified alkali soluble polymer comprises a polymer or copolymer derived from one or more of acrylic acid methacrylic acid, hydroxyethyl methacrylic acid, alkyl preferably C₁ to C₄, methacrylic acid, alkyl preferably C₁ to C₄, acrylic acid, alkyl preferably C₁ to C₄, amino acrylic acids, acrylamides, esters of any such acrylic or methacrylic monomers and ethers of any such acrylic or methacrylic monomers wherein hydrophobic modification is provided by a polyalkylene glycol having one end derivatised with a hydrophobic alcohol. The modified polyalkylene glycol preferably comprises polyethylene glycol, polypropylene glycol or polybutylene glycol, having one end modified by etherification with a hydrophobic alcohol preferably having from 6 to 40 carbon atoms, especially 10 to 30, for example behenyl alcohol.

In a preferred embodiment the hydrophobically modified alkali soluble polymer comprises a copolymer of an acrylate and a methacrylate derivatised with a modified alcohol end-capped polyalkylene glycol as described in the preceding paragraph. An example of an especially preferred polymer is ACULYN 28 and ACULYN 33 ®, available from Dow.

The hydrophobically modified alkali soluble polymer may be neutralised using a pH modifier to a pH of 6 to 8, preferably 6.5 to 7.5 and suitably facilitates optimal thickening of the formulation. pH modifiers known for use in cosmetic or personal care products may be employed in the present composition. Examples of alkaline the pH modifiers include comprises an aminated alcohol, for example aminomethyl propanol and triethanolamine which may also provide a foam stabilizer or surfactant function. Examples of acidic pH modifiers include citric acid and sodium gluconate, which may also provide a humectant function. Examples of suitable bases include AMP Ultra 2000 ® available from Angus Chemical Co, and sodium hydroxide. AMP Ultra 2000 is especially preferred as providing excellent viscosity and stability.

Suitably, the thickening agent is present at a level of 0.1 to 30%, preferably 0.5 to 15%, especially 0.75 to 10% of the cosmetic composition.

A film forming component may be employed instead of or in addition to the thickening agent. The coating composition suitably is film-forming so as to provide an even deposition of the retro-reflective microparticles on the substrate. Examples of suitable film-forming components include trimethoxysiloxysilicates, the COVACRYL™ range of products available from Sensient Cosmetic Technologies, including COVACRYL P12, COVACRYL MT10, COVACRYL MS11 WP, COVACRYL E14 WP; DYNAMX H2O™ STRUCTURE 2001™, CELQUAT L-200™, RESYN 282930™, BALANCE 47™ and BALANCE RCFg™ (available from Akzo Nobel), the FIXATE™ range of products including FREESTYLE™, SUPERHOLD™ G-100, available from Surfachem; TRIQUAT FORCE™ (Tri-K Industries) and LUVISKOL VA™ range of products (BASF).

The coating composition may optionally comprise a modifier which provides a rheology modifier function and/or a humectant function. It is suitably present at a level of 0.1 to 20%, preferably 1 to 10% for example 7% by weight of the coating composition. Where present, the modifier suitably comprises a glycol selected from polyalkylene glycol, preferably polyethylene glycol, polypropylene glycol, or polybutylene glycol, and a C₂ to C₄ alkylene glycol. The polyalkylene glycol preferably comprises units of ethylene glycol, propylene glycol and/or butylene glycol. The polyalkylene glycol may comprise two or more different glycol units and comprises a block copolymer or a random copolymer but preferably comprises one glycol unit, especially ethylene glycol. The polyalkylene glycol suitably comprises from 5 to 100 alkylene glycol units, preferably from 8 to 75 alkylene glycol units, more preferably from 15 to 50 alkylene glycol units, especially from 20 to 40 alkylene glycol units. The polyalkylene glycol may comprise a blend of two or more different polyalkylene glycols. Examples of preferred glycols include PEG 32 and propylene glycol.

Advantageously, the glycol aids dispersion of the retro-reflective microparticles in the composition. Suitably the glycol also acts as a humectant to retain moisture, aiding improved “feel” of the substrate, for example hair fibres.

The thickening agent may also provide a film-forming function or this function may be provided by an additional component of the coating composition. The film-forming component, if present as an additional component in the coating composition is suitably present at a level of 0.5 to 10%, preferably 0.5 to 7%, especially 1 to 5% by weight of the cosmetic composition.

In a preferred embodiment, the coating composition comprises;

A) a thickening agent or film-forming component selected from one or more of:

a. an acrylate copolymer

b. an acrylate crosspolymer-6-2-methyl propan-2-ol;

c. an acrylate/C₁₀₋₃₀ crosspolymer;

d. a cross-linked homopolymer of polyacrylic acid;

e. an acrylate and urethane copolymer;

f. a polyvinylpyrollidone and vinyl acetate copolymer;

g. a copolymer of acrylate and steareth-20 methacrylate

h. Trimethoxysiloxysilicate

i. cetearylalcohol, ceteareth-20

B) a modifier comprising polyethylene glycol, an emulsifier, a conditioner and/or a humectant comprising propylene glycol.

The coating composition is suitably soluble in aqueous solution. In an especially preferred aspect, the invention provides an aqueous hair composition free of goniochromatic colouring agents and which comprises:

i) a transparent coating composition comprising:

A) a thickening agent or film-forming component selected from one or more of:

a. an acrylate copolymer

b. an acrylate crosspolymer-6-2-methyl propan-2-ol;

c. an acrylate/C₁₀₋₃₀ crosspolymer;

d. a cross-linked homopolymer of polyacrylic acid;

e. an acrylate and urethane copolymer;

f. a polyvinylpyrollidone and vinyl acetate copolymer;

g. a copolymer of acrylate and steareth-20 methacrylate

h. Trimethoxysiloxysilicate

i. cetearylalcohol, ceteareth-20

B) a modifier comprising polyethylene glycol, an emulsifier, a conditioner and/or a humectant comprising propylene glycol.

ii) water as a solvent for the transparent coating composition; and

iii) retroreflective particles.

In a further preferred embodiment, the thickening agent is selected from SEPIMAX ZEN and ACULYN 28 and optionally further comprises a film-forming component, preferably, at a level of 1 to 3% by weight.

Suitably, the thickening agent may also provide a fixative effect by aiding adhesion of the microparticles to the substrate without compromising feel or a separate fixative component may be employed in combination with the thickening agent. Suitably the fixative is present at a level of 0.1 to 5%, preferably from 1 to 3.5%.

Where the cosmetic composition is to be formulated as a cream, the coating composition suitably comprises a conditioning agent. Suitable conditioning agents include moisturisers and/or humectants, examples of which include sugar alcohols and glycols. In some embodiments, the conditioning agent is suitably selected from one or more of Keravis PE™ (Croda), Polyderm PPI-CO™ (Aston Chemicals), polyquarternium compounds, suitably in the range polyquaternium-4 to polyquarternium-39 (Aqua (and) Hydrolyzed Vegetable Protein PG-Propyl Silanetriol), D-Panthenol, Glycofilm 1.5P, BELSIL™ PDM 20 (Wacker-Chemie), NATPURE FEEL-M ECO™ (Sensient Cosmetic Technologies).

Optionally, the coating composition may comprise an anionic polysaccharide which may enhance adhesion of the microparticles to the substrate. An example of a preferred anionic polysaccharide is the GLYCOFILM range of products available from Aston Chemicals, for example GLYCOFILM 1.5P Suitably, anionic polysaccharide is present at a level of 0.1-5% by weight, preferably 0.5 to 3%, for example 1% or 1.5%.

The cosmetic composition of the invention is suitably an aqueous composition and suitably comprises water as a solvent for the composition. Preferably the aqueous solvent is selected from water and a mixture of water and a polar organic solvent. Polar organic solvents may be present but are suitably present at a level of less than 20%, preferably less than 10% and desirably less than 5% by weight of the composition of the invention.

Preferably, the solvent is present in the cosmetic composition at a level of 30 to 95%, more preferably 50 to 90%, especially 60 to 80& for example 70% by weight of the composition.

Other optional components may be included in the cosmetic composition as desired including any one or more of a conditioner, a UV stabiliser, a chelating agent, a preservative, conditioning oil, an emulsion a conditioning colour agent and a dye.

In one embodiment, especially suitable for use with hair, the cosmetic composition further comprises a conditioner. Any known hair conditioner may be employed, for example dimethicone and quaternary ammonium salts.

In one embodiment, the composition may further comprise a UV stabiliser. Any known cosmetically approved UV stabilisers may be employed. Preferred UV stabilisers include Benzophenone-4, Avobenzone, Homosalate, Octocrylene, Benzophenone-5, Ethylhexyl Dimethyl PABA, Titanium oxide, Zinc oxide, Phenylbenzimidazole sulfonic acid, PEG-25 PABA, disodium phenyl dibenzimidazole tetrasulfonate.

The composition may also comprise a preservative such as a natural or synthetic antimicrobial. Examples of natural antimicrobials include, carylyl glycol, tea tree oil, potassium sorbate and sorbic acid. Examples of synthetic antimicrobials include phenoxyethanol, benzylalcohol and gluconolactone. Examples of other antimicrobials include benzoic acid, sodium benzoate, sorbic acid, methylparaben and propylparaben.

Where the cosmetic composition is in the form of a styling wax, the composition suitably comprises a structurant comprising a microcrystalline wax.

In a preferred embodiment, the cosmetic composition of the invention comprises:

a thickening agent and/or film forming component selected from a cross-linked polyelectrolyte, and a hydrophobically modified alkali soluble emulsion (HASE), preferably at a level of 0.1 to 30%, more preferably 0.5 to 15%, especially 0.5 to 8% of the cosmetic composition;

a polyalkylene glycol, a polyalkylene glycol derivative or propylene glycol, preferably at a level of 0.1 to 10%, more preferably 0.5 to 7%, especially 1 to 5% of the cosmetic composition;

retro-reflective microparticles at a level of 10 to 50%, preferably 10 to 40%, of the cosmetic composition; and

solvent, preferably water, to 100%.

The cosmetic composition is preferably a hair composition.

The solvent is preferably water but if the composition is to be applied by spray, the water solvent suitably comprises organic components. Preferably, the organic components comprise an alkane, for example butane and pentane, and an alcohol, for example denatured alcohol and ethanol.

In a further preferred embodiment the cosmetic composition may comprise one or more of the following optional components a preservative, a conditioning oil, an emulsion, a conditioning colour agent and a dye.

The cosmetic composition may be colourless or may comprise a colour component. A colour component may be included in the composition to provide a desired aesthetic effect which is observable under normal light conditions or to provide an effect whereby the composition is not readily visible under normal light conditions but which provides an observable colour upon activation with applied directional light.

The microparticles, preferably microspheres, may be tinted with a colour component. The microparticles may be tinted by providing a coloured film or plate within the microparticle such that upon application of directional light, the light Is retro-reflected together with the colour of the tint in the microparticle.

The cosmetic composition may comprise a colour component to provide a desired aesthetic effect while retaining excellent retro-reflectivity. The colour component may be incorporated into the composition as a solution of a dye or as a pigment at any desired level. The colour component may comprise 0.1 to 10% of the cosmetic composition. Preferably, the colour component comprises an aqueous dye.

In one embodiment, the colour component comprises a dye which matches a natural hair colour for example black, blonde, brown and ginger. The colour component may comprise carbon black, iron oxide, graphite synthetic melanin and polydopamine pigment. Examples of suitable black dye or pigments include products available under the trade name MINISO.

The colour component may be any colour for example red, orange, yellow, green, blue, indigo, violet, brown and black. Lighter colours may offer a pearlescent or opalescent effect.

Examples of suitable colour components include the following used alone or in combination: the N,N-Bis(2-Hydroxyethyl)-2-Nitro-p-Phenylenediamine, 4-Amino-3-Nitrophenol, 4-Hydroxypropylamino-3-Nitrophenol, Hydroxyethyl-2-nitro-p-toluidine, Acid black 1, Acid blue 9, Acid green 25, HC Blue No. 12, 3-Nitro-p-Hydroxyethylaminophenol, 2-Amino-6-Chloro-4-Nitrophenol, Acid Red 33, HC Yellow No. 2, HC ORANGE NO. 1, Acid orange 7, acid red 52, acid red 18, curry red, HC YELLOW NO. 13, Basic Yellow 57, HC Red No. 3, Basic Red 76, Basic Red 51, Acid Red 92, Basic Yellow 87, Basic Orange 31, BASIC VIOLET 2, HC Blue No. 16, HC Blue No. 15 Acid Violet 43, Basic Brown 17 and dyes available from Sensient® Cosmetic technologies; Arianor Jade Blue (HC Blue 15), Covalumine fire red AS (CI 15850, Alumina, Triethoxycaprylylsilane), Noir W 699 (CI 20470, Sodium Sulfate), Covalumine astral blue (CI 42090, Alumina, Triethoxycaprylylsilane), Unipure white LC986 FSP (CI 77891, Perfluorooctyl Triethoxysilane, Polyperfluoromethylisopropyl Ether), Unipure red LC3071 (CI 15850, Aluminum Hydroxide), Unipure white LC987 GCA (CI 77891, Sodium Cocoyl Glutamate, Cystine, Lauroyl Arginine), Covarine white WN9787 (CI 77891, Aqua, Glycerin, Xanthan Gum, Sodium Citrate), Covapate Uniwhite LC 9781 (Ricinus Communis (Castor) Seed Oil, CI 77891, Polyhydroxystearic Acid), Unipure white LC981 SGP (CI 77891, Sodium Glycerophosphate), Vert Covasol W7035 (CI 19140, CI 42090), Unipure yellow LC182 ADT-C(CI 77492, Isopropyl Titanium Triisostearate, Bis-PEG-15 Dimethicone/IPDI Copolymer, PEG-2 Soyamine), Unipure black LC989 ADT-C (CI 77499, Isopropyl Titanium Triisostearate, Bis-PEG-15 Dimethicone/IPDI Copolymer, PEG-2 Soyamine), unipure black LC988 FSP (CI 77499, Perfluorooctyl Triethoxysilane, Polyperfluoromethylisopropyl Ether), phat black DC 9206 (mixture of 0161565, 0160725, basic brown 16, acid violet 43, basic red 76, CI 26100), Arianor flash deep black (Basic Blue 99, Basic Brown 16, Acid Violet 43, Basic Red 76, Basic Yellow 57, Basic Brown 17, Polyquaternium-37, Hydrolyzed Yeast Protein). Further examples of suitable dyes include products from the Unipure LC range and Covarine range from Sensient technologies.

Compositions according to the invention may be prepared by mixing the components to provide a homogeneous composition. In a preferred embodiment, the composition is an aqueous composition or an emulsion.

In a further aspect, the invention provides a process for producing a cosmetic composition in the form of an aqueous solution or an aqueous emulsion according to the invention comprising providing a first phase comprising i) water and ii) a thickener and/or a film-forming component, providing a second phase comprising an emulsifier and optionally a conditioner, combining the first phase and second phase at elevated temperature, preferably at least 50° C., more preferably at least 65° C., and homogenising the combined phases and adding retroreflective particles to the homogenised combined phase.

Suitably, the first phase is heated to the desired elevated temperature and the second phase is heated to the desired elevated temperature prior to the first and second phases being combined.

Cosmetic composition according to the invention are especially suitable for use in coating both natural and artificial hair, skin eyelashes and nails. The cosmetic composition may be applied to natural hair, to artificial hair, for example hair extensions, or both.

The cosmetic composition may be formulated as a personal care product for application to keratinous tissue of the human or animal body, for example hair or for application to or incorporation in an artificial substrate. Suitable forms of personal care compositions for topical application include creams, gels, lotions, emulsions, serums, colloids, solutions, suspensions, ointments, milks, sprays, capsules, tablets, liquids, spray-on formulations, brush-on formulations and the like.

In a preferred embodiment, the cosmetic composition is used as a hair product such as a mousse, cream, wax, gel, hair oil or solid hair product optionally dispersed or carried in a liquid, for example a “chalk”.

In a preferred embodiment, the cosmetic composition is in the form of a gel and the composition comprises a gelling agent and a conditioning agent to provide desirable feel.

In another embodiment, the composition is formulated as a spray, for example an aerosol spray and non-aerosol spray and the product comprises a propellant for example a mixture of butane, pentane and denatured alcohol, and the cosmetic composition.

The composition may be formulated as a styling wax.

In another embodiment, the cosmetic composition is suitably formulated a cream and comprises the cosmetic composition and a conditioning agent to provide desirable feel.

Suitably, the cosmetic composition is formulated so as to provide a desired retro-reflective effect upon shining light additional to ambient light or use of flash photograph

The invention is illustrated by the following non-limiting examples. All percentages and parts are parts by weight and all measurements made are at 25° C., unless otherwise stated

### EXAMPLES

### Retroreflective Test and Temporary Application to Substrate

All compositions set out in the Examples below were subjected to testing to demonstrate the retro-reflective effect. Each composition was applied to hair and human skin and photographed both with and without a flash. The compositions all demonstrated a retro-reflective effect when photographed with a flash and no such effect when photographed without the flash. The compositions were all removed from the substrate upon washing with commercially available shampoo for personal use after a maximum of 2 washes.

### Example 1

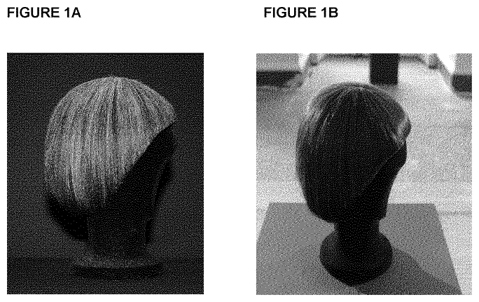

A cosmetic composition according to the invention and as described in Table 2 below was applied to a black real hair wig. The wig was subjected to flash photography and a retro-reflective effect was observed as shown in FIG. 1A. Without the flash light, the wig appeared a natural colour under ambient light conditions as shown in FIG. 1B.

### Example 2

A cosmetic composition according to the invention in gel form was prepared to the composition set out in Table 2 by mixing the listed components.

TABLE 2
Gel

| INCI | Function | Usage level (%) |
|---|---|---|
| Water | Solvent | Q.S. |
| PVP/VA Copolymer | Fixative/film-former | 4.0 |
| Propylene glycol | Humectant | 2.0 |
| Polyacrylate | Viscosity/rheology | 1.0 |
| Crosspolymer-6 | modifier/thickener | |
| PEG-8 | Modifier | 0.2 |
| Phenoxyethanol | Preservative | 1.0 |
| Barium titanate glass, | Retroreflective glass | 30.0 |
| aluminium | microspheres (visual | |
| | effect) | |

### Example 3

A cosmetic composition according to the invention in the form of an aerosol spray was prepared to the composition set out in Table 3 by mixing the listed components.

TABLE 3
Aerosol spray

| INCI | Function | Usage level (%) |
|---|---|---|
| Butane | Solvent | Q.S. |
| Alcohol denat. | Solvent | 20.0 |
| Pentane | Solvent | 10.0 |
| Trimethoxysiloxysilicate | Film former/fixative | 4.0 |
| Triethoxycaprylylsilane | Dispersing aid | 0.2 |
| Barium titanate glass, | Retroreflective glass | 20.0 |
| aluminium | microspheres (visual | |
| | effect) | |

### Example 4

A cosmetic composition according to the invention in the form of a styling wax was prepared to the composition set out in Table 4 by mixing the listed components.

TABLE 4
Styling wax

| INCI | Function | Usage level (%) |
|---|---|---|
| Water | Solvent | Q.S. |
| Acrylates/C10-30 Alkyl | Viscosity/rheology | 0.5 |
| Acrylate Crosspolymer | modifier/thickener/film- | |
| | former | |
| Propylene glycol | Humectant | 10.0 |
| Cetearyl alcohol, | Emulsifier/viscosity | 5.0 |
| ceteareth-20 | modifier | |
| Microcrystalline waxes | Structurant | 15.0 |
| PVP/VA Copolymer | Fixative | 5.0 |
| PEG-12 dimethicone | Emollient/conditioner | 5.0 |
| NaOH sol. (25% w/w) | pH modifier | As required |
| Phenoxyethanol | Preservative | 1.0 |
| Barium titanate glass, | Retroreflective glass | 40.0 |
| aluminium | microspheres (visual | |
| | effect) | |

### Example 5

A cosmetic composition according to the invention in the form of a cream was prepared to the composition set out in Table 5 by mixing the listed components.

TABLE 5
Cream

| INCI | Function | Usage level (%) |
|---|---|---|
| Water | Solvent | Q.S. |
| Butyrospermum parkii | Emollient | 4.0 |
| butter | | |
| Cetrimonium chloride | Conditioning agent | 1.0 |
| Cetearyl alcohol, | Emulsifier/viscosity | 4.0 |
| ceteareth-20 | modifier | |
| Coconut oil | Emollient | 1.0 |
| Acrylates/C10-30 Alkyl | Viscosity/rheology | 2.0 |
| Acrylate Crosspolymer | modifier | |
| Citric acid | pH modifier | As required |
| Phenoxyethanol | Preservative | 1.0 |
| Barium titanate glass, | Retroreflective glass | 40.0 |
| aluminium | microspheres (visual | |
| | effect) | |

pH =3.5 - 4.5

### Example 6—Emulsion and Conditioner

A cosmetic composition according to the invention in the form of an emulsion was prepared to the composition set out in Table 6 below. Phase A was prepared by adding CARBOPOL ULTREZ 21 to the water, allowing to hydrate and then adding AMP to neutralise until a clear gel formed. The mixture was then heated to 70° C. Phase B was prepared by combining the components in a beaker and heating to 70° C. until the waxes had melted. Phase B was then added to Phase A at 70° C. and homogenised at 4000 rpm and cooled to 40° C. The components of Phase C were then added to the Phase A/Phase B mixture and homogenised. The beads of Phase D were then added to the mixture with mixing. The final composition had a pH of 4 to 4.5.

TABLE 6

| Phase | Trade Name | INCI | Function | Level (%) |
|---|---|---|---|---|
| A | Water | Aqua | Solvent | 62.18% |
| A | Carbopol | Acrylates/C10-30 | Thickener | 0.30% |
| | Ultrez 21 | Alkyl Acrylate | | |
| | | Crosspolymer | | |
| A | AMP Ultra | Aminomethyl- | Neutraliser for | |
| | c100 | propanol | Thickener | 0.02% |
| B | Dimethicone | Dimethicone | Hair | |
| | | | conditioning | 1.50% |
| B | Prolipid 141 | Glyceryl Stearate | Emulsifier | 5.00% |
| | Lamellar | (and) Behenyl | | |
| | | Alcohol (and) | | |
| | | Palmitic Acid (and) | | |
| | | Stearic Acid (and) | | |
| | | Lecithin (and) | | |
| | | Lauryl Alcohol | | |
| | | (and) Myristyl | | |
| | | Alcohol (and) | | |
| | | Cetyl Alcohol | | |
| C | Phenoxyethanol | Phenoxyethanol | preservative | 1.00% |
| D | Retroreflective | Glass, Aluminium | Retroreflective | |
| | beads | | Effect | 30.00% |
| | | | | 100.00% |

The composition of Example 6 was applied to hair and provided excellent retro-reflective effect when subjected to flash photography, an even coating on the hair and feel due to the conditioner. The hair was subjected to a standard washing process employing a commercially available shampoo for personal use. The hair was substantially free from the composition after 2 washes.

1. A cosmetic composition for providing a retro-reflective effect to incident light, comprising retroreflective microparticles and a transparent coating composition comprising a thickening agent and/or a film-forming component and being substantially free of goniochromatic colouring agents.

2. A cosmetic composition according to claim 1 wherein the microparticles comprise transparent or translucent microspheres.

3. A cosmetic composition according to claim 1 wherein the microparticles comprise a glass.

4. A cosmetic composition according to claim 3 wherein the microparticles comprise a metal titanate.

5. A cosmetic composition according to claim 1 wherein the microparticles provide retro-reflectivity and specular reflection.

6. A cosmetic composition according to claim 1 wherein the microparticles comprise a reflective coating.

7. A cosmetic composition according to claim 6 wherein the reflective coating comprises a metal or a metallic compound.

8. A cosmetic composition claim 1 wherein the retro-reflective particles have a refractive index of 1.5 to less than 3.0.

9. A cosmetic composition according to claim 1 wherein the microparticles have a particle size of less than 100 microns.

10. (canceled)

11. (canceled)

12. A cosmetic composition according to claim 1 wherein the composition is removable from a substrate to which it is applied in 5 or fewer standard washes.

13. (canceled)

14. A cosmetic composition according to claim 1 further comprising one or more of a polymeric binder, a gelling agent, a conditioning agent, a glossing agent and a shine agent.

15. (canceled)

16. A cosmetic composition according to claim 1 wherein the thickening agent is selected from a cross-linked polyelectrolyte and a hydrophobically modified alkali soluble polymer.

17. A cosmetic composition according to claim 1 wherein the thickening agent is selected from a polyacrylate polymer, cross-polymers and copolymers of (poly)acrylates and polyacrylic acids, for example polyacrylate crosspolymer-6-2-methyl propan-2-ol and acrylates/C10-30 alkyl acrylate crosspolymer, alkyl acrylates, urethane acrylates, hydrophilically modified starch, polyvinyl pyrollidone/vinyl acetate copolymers, polyquaternium compounds and cetearyl alcohol, ceteareth-20.

18. (canceled)

19. A cosmetic composition according to claim 1 wherein the coating composition comprises;
A) a thickening agent at a level of 0.1 to 30% and a film-forming component at a level of 0.5 to 10% and
B) a modifier comprising polyethylene glycol, an emulsifier, a conditioner and/or a humectant comprising propylene glycol.

20. A cosmetic composition according to claim 1 further comprising a colour component selected from a dye and a pigment.

21. A cosmetic composition according to claim 20 wherein the colour component further comprises a colour component selected from a black, brown or blonde component.

22. (canceled)

23. A coating product for a substrate comprising a cosmetic composition according to claim 1 in the form of a cream, oil, wax, spray, emulsion or gel.

24. (canceled)

25. A process of providing a keratinous substrate, with the capability of exhibiting a retro-reflective effect comprising applying a cosmetic composition according to claim 1 to the substrate wherein the cosmetic composition provides a temporary capability of exhibiting a retro-reflective effect by being removable from the substrate by subjecting to standard washing, and being removable from the substrate in 5 or fewer standard washes.

26. (canceled)

27. A cosmetic cream composition for providing a retro-reflective effect to incident light, comprising:
i) retroreflective microparticles;
ii) a conditioning agent and/or emollient;
iii) a transparent coating composition comprising a thickening agent at a level of 0.1 to 30% and/or a film-forming component at a level of 0.5 to 10% by weight of the cream composition; and
iv) being substantially free of goniochromatic colouring agents.
